(19) United States
(12) Patent Application Publication
(10) Pub. No.: US 2008/0172106 A1
(43) Pub. Date: Jul. 17, 2008

(54) OSTEOGENIC STIMULUS DEVICE, KIT AND METHOD OF USING THEREOF

(76) Inventors: William J. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/622,089

(22) Filed: Jan. 11, 2007

(51) Int. Cl. A61N 1/05

(52) U.S. Cl. 607/51

(57) ABSTRACT

An osteogenic stimulus device having at least a pair of electrode coupled to a working circuit is presented for use in applying a therapeutic electrical signal to, in, around, and across an osseous structure to aid in the healing and bone growth process in which the therapeutic electrical signal is a function of the electrical impedance of the fractured bone. The working circuit of the osteogenic stimulus device includes an impedance measurement sub-circuit, a therapeutic application sub-circuit, and a switch sub-circuit. The kit for the osteogenic stimulus device includes the uncoupled components of the device. The method of using the osteogenic stimulus device includes the acts of activating, applying, assessing, coupling, joining, mounting, obtaining, and removing.

### FIELD OF THE INVENTION

The present invention relates generally to medical devices and more particularly to an osteogenic stimulus device for use in stimulating, fusing and healing osseous structures, and tissue in the presence of an applied therapeutic electrical signal in which the applied therapeutic electrical signal is variably responsive to an electrical impedance measured across the osseous structure.

### DESCRIPTION OF THE PRIOR ART

The utilization of electric phenomenon to aid in expediting the healing of bone fractures or bone defects in a patient is well known in the art and has been the subject of numerous publications. Accordingly, a wide variety of electrical medical devices is currently available on the commercial market and an even larger number of these types of devices are known in the art of electrical medical devices, for example, the method for aiding formation of bone forming material disclosed by Kraus in U.S. Pat. No. 3,783,880; the constant current power pack for bone healing and method of use disclosed by Brighton et al. in U.S. Pat. No. 3,842,841; the tissue growth control apparatus and method disclosed by Greatbatch in U.S. Pat. No. 4,313,438, the bone growth stimulator disclosed by Jeffcoat and Wickham in U.S. Pat. No. 4,333,469; the bone and tissue healing device including a special electrode assembly and method disclosed by Christensen in U.S. Pat. No. 4,461,300; the combined tissue/bone growth stimulator and external fixation device disclosed by Tepper and Bryant in U.S. Pat. No. 6,678,562; the method and device for treating osteoarthritis, cartilage disease, defects and injuries in the human knee disclosed by Brighton and Pollack in U.S. Pat. No. 7,022,506; and the combination electrical stimulating and infusion medical device and method disclosed by Vilims in U.S. Pat. Publ. No. 2006/0155343. On treating fractured, injured and diseased osseous structures, such devices have ranged in size and complexity from large, bulky systems feeding electrical pulses by conductors extending through the skin.

While all of the above-described devices fulfill their respective, particular objectives and requirements, the aforementioned patents do not describe an osteogenic stimulus device having a pair of electrodes coupled to a working circuit that has an impedance measurement sub-circuit, a therapeutic application sub-circuit, and a switch sub-circuit.

This combination of elements would specifically match the user's particular individual needs of making it possible to provide a convenient and effective means for applying a therapeutic electrical signal on a fractured bone to aid in the healing process in which the applied therapeutic electrical signal is variably responsive to an electrical impedance measured across a portion of the fractured bone. The above-described patents make no provision for an device having a pair of electrodes coupled to a working circuit that has an impedance measurement sub-circuit, a therapeutic application sub-circuit, and a switch sub-circuit so that a therapeutic electrical signal can be applied to the fracture in which the therapeutic electrical signal is variably responsive to a measured electrical impedance across a portion of that fracture. Therefore, a need exists for a new and improved osteogenic stimulus device having a pair of electrodes coupled to a working circuit having an impedance measurement sub-circuit, a therapeutic application sub-circuit, and a switch sub-circuit for use in applying a variable therapeutic electrical signal across a fractured bone to aid in the healing process, in which the therapeutic electrical signal is dependent upon a measured impedance across a section of the fractured bone.

The osteogenic stimulus device includes a pair of electrodes coupled to a working circuit composed of an impedance measurement sub-circuit, a therapeutic application sub-circuit, and a switch sub-circuit in which configured to apply a therapeutic electrical signal on compromised bone to aid in the healing process of the bone, in which the therapeutic electrical signal is dependent upon on an empirically measured electrical impedance of the bone. In this respect, the osteogenic stimulus device according to the present invention substantially departs from the conventional concepts and designs of the prior art, and in doing so provides an medical device, kit and method primarily developed for the purpose of providing a convenient and effective means for applying a therapeutic electrical signal across a fractured bone to aid in the healing process in which the therapeutic electrical signal is responsive to a measured impedance across the fractured bone.

### SUMMARY OF THE INVENTION

The osteogenic stimulus present device, kit and method of using, according to the principles of the present invention, overcomes a number of the shortcomings of the prior art by providing an osteogenic stimulus device having a pair of electrodes and a working circuit is presented for use in applying a therapeutic electrical signal across a fractured bone to aid in the healing process in which the therapeutic electrical signal is variably dependent upon a measured impedance across the healing bone. The working circuit of the osteogenic stimulus device includes an impedance measurement sub-circuit, a therapeutic application sub-circuit, and a switch sub-circuit. The kit for the osteogenic stimulus device includes the uncoupled components of the device. The method of using the osteogenic stimulus device includes the acts of activating, applying, assessing, coupling, joining, mounting, obtaining, and removing.

In view of the foregoing disadvantages inherent in the known type osteogenic stimulus devices and method for use now present in the prior art, the present invention provides an improved osteogenic stimulus device, which will be described subsequently in great detail, is to provide a new and improved osteogenic stimulus device which is not anticipated, rendered obvious, suggested, or even implied by the prior art, either alone or in any combination thereof.

To attain this, the present invention essentially comprises an osteogenic stimulus device having the interconnected elements of a first electrode, a second electrode and a working circuit is presented for use in applying a therapeutic electrical signal across a fractured bone to aid in the healing process in which the therapeutic electrical signal is functionally dependent upon a measured impedance across a portion of the fractured bone. The working circuit of the osteogenic stimulus device includes an impedance measurement sub-circuit, a therapeutic application sub-circuit, and a switch sub-circuit in which the working circuit is configured to measure an electrical impedance and to subsequently apply a therapeutic electrical signal as a function of the measured electrical impedance.

There has thus been outlined, rather broadly, the more important features of the invention in order that the detailed description thereof that follows may be better understood, and in order that the present contribution of the art may be better appreciated.

The invention may also include a number of optional elements, such as an ion probe, a power supply, and an external control system.

Numerous objects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved osteogenic stimulus device that has many of the advantages of the prior osteogenic stimulus devices and minimizing a number of their disadvantages.

It is another aspect of the present invention to provide a new and improved osteogenic stimulus device that may be easily and efficiently manufactured and marketed.

An even further aspect of the present invention is to provide a new and improved osteogenic stimulus device that has a low cost of manufacture with regard to both materials and labor, and which accordingly is then susceptible of low prices of sale to the consuming public, thereby making osteogenic stimulus devices economically available to the buying public.

Still another aspect of the present invention is to provide an osteogenic stimulus device that is configured to measure an impedance across a fractured bone and is configured to applying a therapeutic electrical signal across the fractured bone in which the therapeutic electrical signal is a function of the fractured bone impedance.

Even still another aspect of the present invention is to provide a kit for an osteogenic stimulus device comprising the uncoupled elements of a first electrode, a second electrode, a working circuit configured to be coupled to the first and second electrodes, and a power supply 30. The working circuit of the kit comprises an impedance measurement sub-circuit, a therapeutic application sub-circuit, and a switch sub-circuit.

Lastly, it is an aspect of the present invention to provide a new and improved method of using an osteogenic stimulus device 10 to aid in healing a fractured bone comprises the acts of activating, applying, assessing, coupling, joining, mounting, obtaining, and removing.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is perspective view of an osteogenic stimulus device constructed in accordance with the principles of the present invention;

FIG. 2 is a perspective view of another embodiment of the osteogenic stimulus device;

FIG. 3 is a perspective view of another embodiment of the osteogenic stimulus device;

FIG. 4 is a stylized electronic schematic of an osteogenic stimulus device of the present invention;

FIG. 5 is a logical schematic embodiment of an osteogenic stimulus device of the present invention;

FIG. 6 is another logical schematic embodiment of an osteogenic stimulus device of the present invention;

FIG. 7 is a perspective view of another embodiment of an osteogenic stimulus device of the present invention;

FIG. 8 is side view of one embodiment of an osteogenic stimulus device of the present invention mounted within a fractured bone; and

FIG. 9 is side view of another embodiment of an osteogenic stimulus device of the present invention mounted within a fractured bone.

The same reference numerals refer to the same parts throughout the various figures.

### DETAILED DESCRIPTION OF THE PREFERRED EMBODIMENT

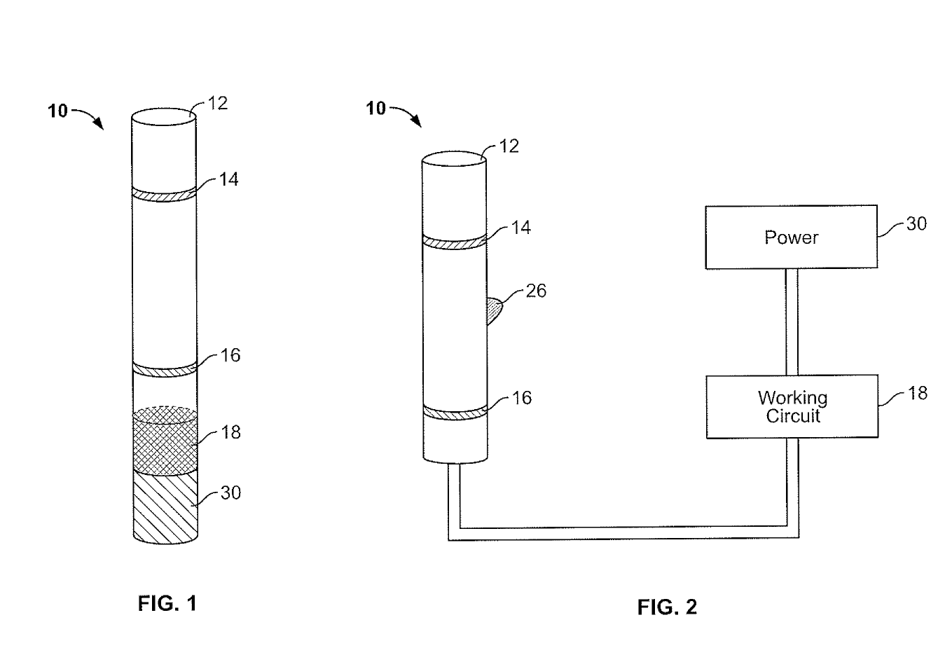

Referring now to the drawings, and in particular FIG. 1 to FIG. 9, which depict various embodiments of the present invention of the osteogenic stimulus device 10.

One preferred embodiment the osteogenic stimulus device 10 comprises a housing 12, a first electrode 14, a second electrode 16, and a working circuit 18. The first electrode 14 is attached to the housing 12. The second electrode 16 is attached to the housing 12. The working circuit 18 is coupled to the first and second electrodes (14 and 16, respectively) in which the working circuit 18 comprises an impedance measurement sub-circuit 20, a therapeutic application sub-circuit 22, and a switch sub-circuit 24. The impedance measurement sub-circuit 20 is coupled to the first and second electrodes (14 and 16, respectively) in which the impedance measurement sub-circuit 20 is configured to measure an electrical impedance across the first and second electrodes (14 and 16, respectively). The therapeutic application sub-circuit 22 is coupled to the first and second electrodes (14 and 16, respectively) and coupled to the impedance measurement sub-circuit 20 in which the therapeutic application sub-circuit 22 is configured to apply a therapeutic electrical signal across the first and second electrodes (14 and 16, respectively) in response to the measured electrical impedance. The switch sub-circuit 24 is coupled to the impedance measurement sub-circuit 20 and to the therapeutic application sub-circuit 22 in which the switch sub-circuit 24 is configured to enable alternately the impedance measurement sub-circuit 20 and the therapeutic application sub-circuit 22 during a cycle period.

The working circuit 18 of the device 10 may be any commercially available working circuit 18. One preferred embodiment of the working circuit 18 is that it is a microprocessor working circuit 18.

The impedance measurement sub-circuit 20 may be configured to measure any electrical impedance, such as a DC resistive electrical impedance, AC frequency dependant complex electrical impedance or even a DC conductance in mhos.

The therapeutic application sub-circuit 22 of the device 10 may be configured to apply any known therapeutic electrical signal. One preferred embodiment is that the therapeutic application sub-circuit 22 is configured to set a DC voltage of the therapeutic electrical signal in response to the measured electrical impedance. Another preferred embodiment is that the therapeutic application sub-circuit 22 sets a DC voltage of the therapeutic electrical signal in response to the measured electrical impedance between 1 to 2 volts. Still another preferred embodiment is that the therapeutic application sub-circuit 22 sets a DC current of the therapeutic electrical signal in response to the measured electrical impedance. Yet another preferred embodiment is that the therapeutic application sub-circuit 22 sets the therapeutic electrical signal in response to the measured electrical impedance between 5 to 20 microamps. Even yet another preferred embodiment is that the therapeutic application sub-circuit 22 sets an AC voltage of the therapeutic electrical signal in response to the measured electrical impedance. Finally another preferred embodiment is that the therapeutic application sub-circuit 22 sets an AC current of the therapeutic electrical signal in response to the measured electrical impedance.

The switch sub-circuit 24 of the device 10 may impose any a cycle period duration. One preferred embodiment is that the switch sub-circuit 24 imposes having a cycle period duration of at least one minute in which the switch sub-circuit 24 enables the therapeutic application sub-circuit 22 to apply the therapeutic electrical signal during at least 90% of the cycle period duration and the switch sub-circuit 24 enabling the impedance measurement sub-circuit 20 to measure the electrical impedance less than 10% of the cycle period duration. Another preferred embodiment is that the switch sub-circuit 24 imposes having a cycle period duration of at least one minute in which the switch sub-circuit 24 enables the therapeutic application sub-circuit 22 to apply the therapeutic electrical signal during at least 99% of the cycle period duration and the switch sub-circuit 24 enabling the impedance measurement sub-circuit 20 to measure the electrical impedance less than 1% of the cycle period duration.

The housing 12 of the device 10 may have any known design of housing 12 as long as it is able to have the first electrode 14, the second electrode 16 attached to it. One preferred embodiment of the housing 12 is that it pedicle screw housing 12. Another preferred embodiment of the housing 12 is that the working circuit 18 is attached to the housing 12. Yet another preferred embodiment of the housing 12 is that the power supply 30 is attached to the housing 12.

An optional ion probe 26 and an ion monitoring sub-circuit 28 may be added to the device 10. The ion probe may be attached to the housing 12 or may be separate from the housing 12. The optional ion monitoring sub-circuit 28 is coupled to the ion probe 26 in which the ion monitoring sub-circuit 28 is configured to measure an ion signal from the ion probe 26, wherein the ion signal is proportional to an ion accumulation phenomenon at an interface between the ion probe 26 and its surrounding. The ion monitoring sub-circuit 28 is coupled to the switch sub-circuit 24 in which the switch sub-circuit 24 is optionally configured to disable the therapeutic application sub-circuit 22 from applying the therapeutic electrical signal when the ion signal from the ion probe 26 is above a set threshold value. The optional ion probe 26 may be any commercially available ion probe 26 such ion probes 26 selected from the group consisting of a hydronium ion probe 26, a hydroxide ion probe 26, a calcium ion probe 26, a fluoride ion probe 26, a chloride ion probe 26, a potassium ion probe 26, and a phosphate ion probe 26. In the case where the ion probe 26 is a hydronium ion probe 26, one preferred setting for the threshold value of the ion signal of the hydronium ion probe 26 corresponds to a pH of about 8.

An optional power supply 30 may be added to the device 10 in which the power supply 30 is coupled to the working circuit 18.

An optional reporting sub-circuit 32 may be added working circuit 18 of the device 10. One preferred embodiment of the reporting sub-circuit 32 is that it is coupled to the input impedance measurement sub-circuit 20, to the therapeutic application sub-circuit 22 and to the switch sub-circuit 24 wherein the reporting sub-circuit 32 configured to report data information corresponding to the measured electrical impedance, to the applied electrical signal and to the cycle period, respectively. Another preferred embodiment of the reporting sub-circuit 32 is that is that it is coupled to the input impedance measurement sub-circuit 20, to the therapeutic application sub-circuit 22, to the switch sub-circuit 24, and to the ion probe 26 sub-circuit wherein the reporting sub-circuit 32 configured to report data information corresponding to the measured electrical impedance, to the applied electrical signal, to the cycle period and to the ion signal, respectively.

An optional receiver sub-circuit 34 may be added working circuit 18 of the device 10. One preferred embodiment is that the receiver sub-circuit 34 is coupled to the therapeutic application sub-circuit 22 and to the switch sub-circuit 24 in which the receiver sub-circuit 34 configured to receive directive information corresponding to adjust the therapeutic electrical signal and to adjust the cycle period.

An optional external control system 36 may be added to the device 10. One preferred embodiment of the optional external control system 36 is that it is configured to be coupled to the receiver sub-circuit 34 in which the external control system 36 is configured to send directive information corresponding to adjust the therapeutic electrical signal and to adjust the cycle period.

Another preferred embodiment of the osteogenic stimulus device 10 comprises a housing 12, a plurality of opposing electrode pairs 38, and a working circuit 18. The plurality of opposing electrode pairs 38 is attached to the housing 12. The working circuit 18 is coupled to each opposing electrode pairs 38, in which the working circuit 18 comprises an impedance measurement sub-circuit 20, a therapeutic application sub-circuit 22 and a switch sub-circuit 24. The impedance measurement sub-circuit 20 is coupled to each opposing electrode pair 38 in which the impedance measurement sub-circuit 20 is configured to measure a corresponding electrical impedance across each corresponding opposing electrode pair 38. The therapeutic application sub-circuit 22 is coupled to each opposing electrode pair 38 and coupled to the impedance measurement sub-circuit 20 in which the therapeutic application sub-circuit 22 is configured to apply a corresponding therapeutic electrical signal across each corresponding opposing electrode pair 38 in response to the corresponding measured electrical impedance. The switch sub-circuit 24 is coupled to the impedance measurement sub-circuit 20 and to the therapeutic application sub-circuit 22 in which the switch sub-circuit 24 is configured to enable alternately the impedance measurement sub-circuit 20 and the therapeutic application sub-circuit 22 during a cycle period.

Still another preferred embodiment of the osteogenic stimulus device 10 comprises a first electrode 14; a second electrode 16; and a working circuit 18. The working circuit 18 is coupled to the first and second electrodes (14 and 16, respectively), in which the working circuit 18 comprises an impedance measurement sub-circuit 20, an therapeutic application sub-circuit 22, and a switch sub-circuit 24. The impedance measurement sub-circuit 20 is coupled to the first and second electrodes (14 and 16, respectively) in which the impedance measurement sub-circuit 20 is configured to measure an electrical impedance across the first and second electrodes (14 and 16, respectively). The therapeutic application sub-circuit 22 is coupled to the first and second electrodes (14 and 16, respectively) and is coupled to the impedance measurement sub-circuit 20 in which the therapeutic application sub-circuit 22 is configured to apply a therapeutic electrical signal across the first and second electrodes (14 and 16, respectively) in response to the measured electrical impedance. The switch sub-circuit 24 is coupled to the impedance measurement sub-circuit 20 and is coupled to the therapeutic application sub-circuit 22 in which the switch sub-circuit 24 is configured to enable alternately the impedance measurement sub-circuit 20 and the therapeutic application sub-circuit 22 during a cycle period.

One preferred embodiment of a kit for an osteogenic stimulus device 10 comprises the uncoupled elements of a first electrode 14, a second electrode 16, a working circuit 18 configured to be coupled to the first and second electrodes (14 and 16, respectively) and a power supply 30. The working circuit 18 comprises an impedance measurement sub-circuit 20, a therapeutic application sub-circuit 22, and a switch sub-circuit 24. The impedance measurement sub-circuit 20 is coupled to the first and second electrodes (14 and 16, respectively) in which the impedance measurement sub-circuit 20 is configured to measure an electrical impedance across the first and second electrodes (14 and 16, respectively). The therapeutic application sub-circuit 22 is coupled to the first and second electrodes (14 and 16, respectively) and is coupled to the impedance measurement sub-circuit 20 wherein the therapeutic application sub-circuit 22 configured to apply a therapeutic electrical signal across the first and second electrodes (14 and 16, respectively) in response to the measured electrical impedance. The switch sub-circuit 24 is coupled to the impedance measurement sub-circuit 20 and is coupled to the therapeutic application sub-circuit 22 in which the switch sub-circuit 24 is configured to enable alternately the impedance measurement sub-circuit 20 and the therapeutic application sub-circuit 22 in a cycle period. The power supply 30 is capable of being coupled to the working circuit 18.

One preferred embodiment of a method of using a kit for an osteogenic stimulus device 10 to aid in healing a fractured bone 40, the method comprising the acts of activating, applying, assessing, connecting, coupling, joining, linking, mounting, obtaining, removing, and securing. The obtaining act comprises obtaining the kit for an osteogenic stimulus device 10, said kit comprising: a first electrode 14; a second electrode 16; and a working circuit 18 configured to be coupled to the first and second electrodes (14 and 16, respectively), the working circuit 18 comprising: an impedance measurement sub-circuit 20 coupled to the first and second electrodes (14 and 16, respectively) wherein the impedance measurement sub-circuit 20 configured to measure an electrical impedance across the first and second electrodes (14 and 16, respectively); an therapeutic application sub-circuit 22 coupled to the first and second electrodes (14 and 16, respectively) and coupled to the impedance measurement sub-circuit 20 wherein the therapeutic application sub-circuit 22 configured to apply a therapeutic electrical signal across the first and second electrodes (14 and 16, respectively) in response to the measured electrical impedance; and a switch sub-circuit 24 coupled to the impedance measurement sub-circuit 20 and to the therapeutic application sub-circuit 22 wherein the switch sub-circuit 24 configured to enable alternately the impedance measurement sub-circuit 20 and the therapeutic application sub-circuit 22 in a cycle period; and a power supply 30 coupled to the working circuit 18. The joining act comprises joining together the fractured bone 40. The mounting act comprises mounting the first electrode 14 into a first section of the joined fractured bone 40. The securing act comprises securing the second electrode 16 into a second section of the joined fractured bone 40. The coupling act comprises coupling the first and second electrodes (14 and 16, respectively) to the working circuit 18. The connecting act comprises connecting together the working circuit 18 with the power supply 30. The applying act comprises applying a test electrical signal across the first and second electrodes (14 and 16, respectively). The assessing act comprises assessing whether or not an innervated muscle has been stimulated by the applied test electrical signal. The removing act comprises removing the first or second electrode 16s from the joined fractured bone 40 when the innervated muscle has been assessed to have been stimulated by the applied test electrical signal. The linking act comprises linking operatively together the power supply 30 to the working circuit 18. The activating act comprises activating switch sub-circuit 24 to enable the working circuit 18 to measure the electrical impedance across the first and second electrodes (14 and 16, respectively) and to enable the therapeutic application sub-circuit 22 to apply a therapeutic electrical signal across the first and second electrodes (14 and 16, respectively) in response to the measured electrical impedance.

One preferred method of using an osteogenic stimulus device 10 to aid in healing a fractured bone 40 comprises the acts of activating, applying, assessing, coupling, joining, mounting, obtaining, and removing. The obtaining act comprises obtaining the osteogenic stimulus device 10 comprising: a housing 12; a first electrode 14 attached to the housing 12; a second electrode 16 attached to the housing 12; and a working circuit 18 coupled to the first and second electrodes (14 and 16, respectively), the working circuit 18 comprising: an impedance measurement sub-circuit 20 coupled to the first and second electrodes (14 and 16, respectively) wherein the impedance measurement sub-circuit 20 configured to measure an electrical impedance across the first and second electrodes (14 and 16, respectively); a therapeutic application sub-circuit 22 coupled to the first and second electrodes (14 and 16, respectively) and coupled to the impedance measurement sub-circuit 20 wherein the therapeutic application sub-circuit 22 configured to apply a therapeutic electrical signal across the first and second electrodes (14 and 16, respectively) in response to the measured electrical impedance; and a switch sub-circuit 24 coupled to the impedance measurement sub-circuit 20 and to the therapeutic application sub-circuit 22 wherein the switch sub-circuit 24 configured to enable alternately the impedance measurement sub-circuit 20 and the therapeutic application sub-circuit 22 in a cycle period. The joining act comprises joining together the fractured bone 40. The mounting act comprises mounting the housing 12 to the joined fractured bone 40. The applying act comprises applying a test electrical signal across the first and second electrodes (14 and 16, respectively). The assessing act comprises assessing whether or not an innervated muscle has been stimulated by the applied test electrical signal. The removing act comprises removing the mounted housing 12 to the joined fractured bone 40 when the innervated muscle been assessed to have been stimulated by the applied test electrical signal. The coupling act comprises coupling together the power supply 30 to the working circuit 18. The activating act comprises activating switch sub-circuit 24 to enable the working circuit 18 to measure the electrical impedance across the first and second electrodes (14 and 16, respectively) and to enable the therapeutic application sub-circuit 22 to apply a therapeutic electrical signal across the first and second electrodes (14 and 16, respectively) in response to the measured electrical impedance.

Referring now to FIG. 1 that depicts a perspective view of an osteogenic stimulus device 10 comprising a housing 12, a first electrode 14, a second electrode 16, a working circuit 18 and a power supply 30 showing all of the elements attached directly to the housing 12

Referring now to FIG. 2 which depicts perspective view of another embodiment of the osteogenic stimulus device 10 comprising a housing 12, a first electrode 14, a second electrode 16, a working circuit 18, ion probe 26, and a power supply 30. The first and second electrode (14 and 16, respectively), and the ion probe 26 are shown attached directly to the housing 12. The working circuit 18, and the power supply 30 are shown not directly attached to the housing 12.

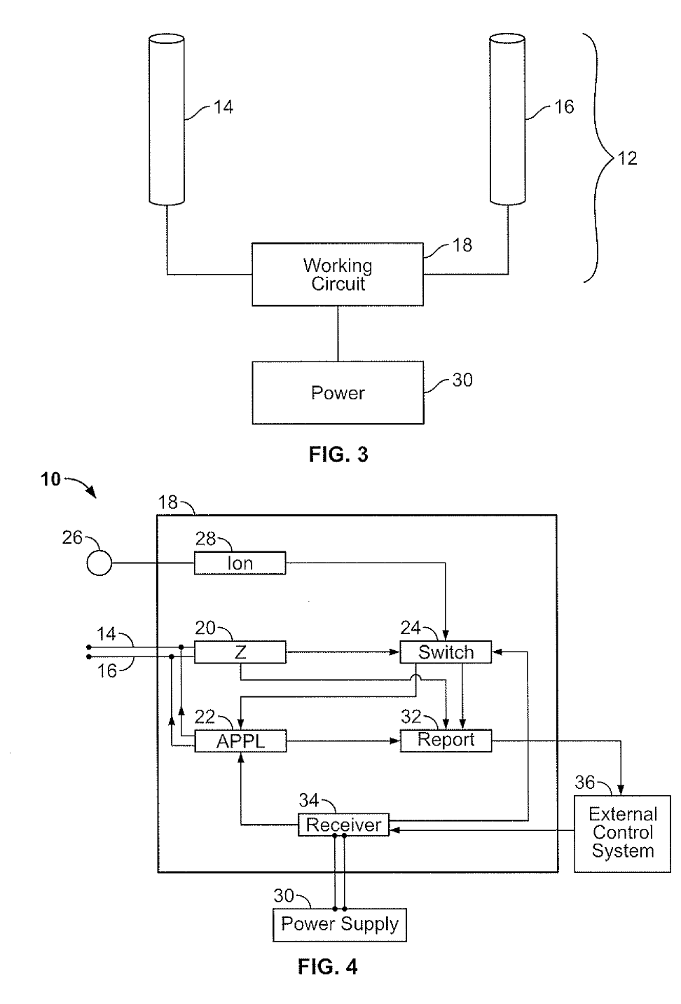

Referring now to FIG. 3 which depicts a perspective view of another embodiment of the osteogenic stimulus device comprising a first electrode 14, a second electrode 16, a working circuit 18, and a power supply 30 in which none of the respective elements are directly attached to each other.

Referring now to FIG. 4 which is a stylized electronic schematic of embodiment of an osteogenic stimulus device 10 of the present invention showing a first electrode 14, a second electrode 16, a working circuit 18, a power supply 30 and an external control system 36. The working circuit 18 is shown coupled to the first and second electrodes (14 and 16, respectively), to the ion probe 26, to the power supply 30 and to the external an external control system 36. The working circuit 18 is shown comprising the interconnected components of a impedance measurement sub-circuit 20, a therapeutic application sub-circuit 22, a switch sub-circuit 24, an ion monitoring sub-circuit 28, a reporting sub-circuit 32, and a receiver sub-circuit 34.

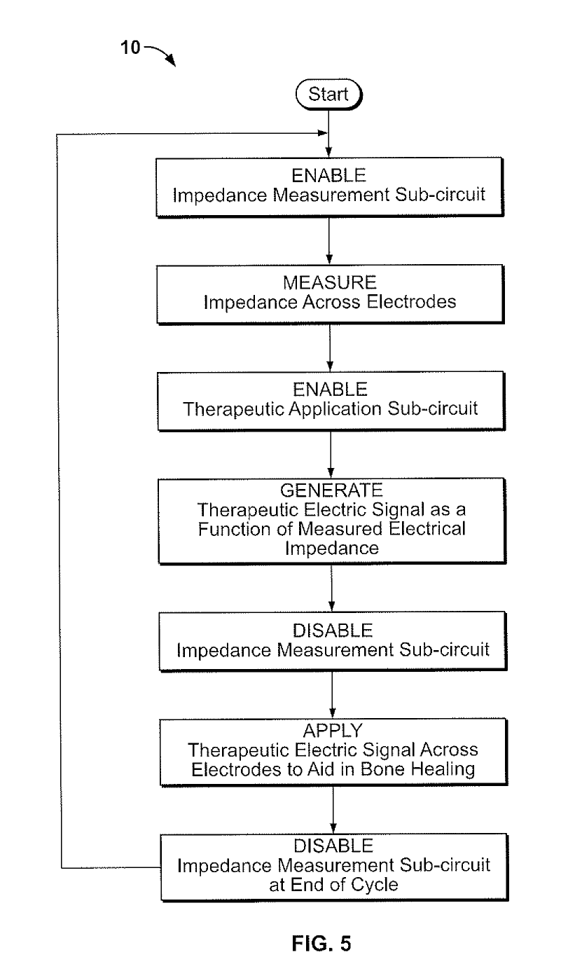

Referring now to FIG. 5 is a logical schematic embodiment of an osteogenic stimulus device of the present invention.

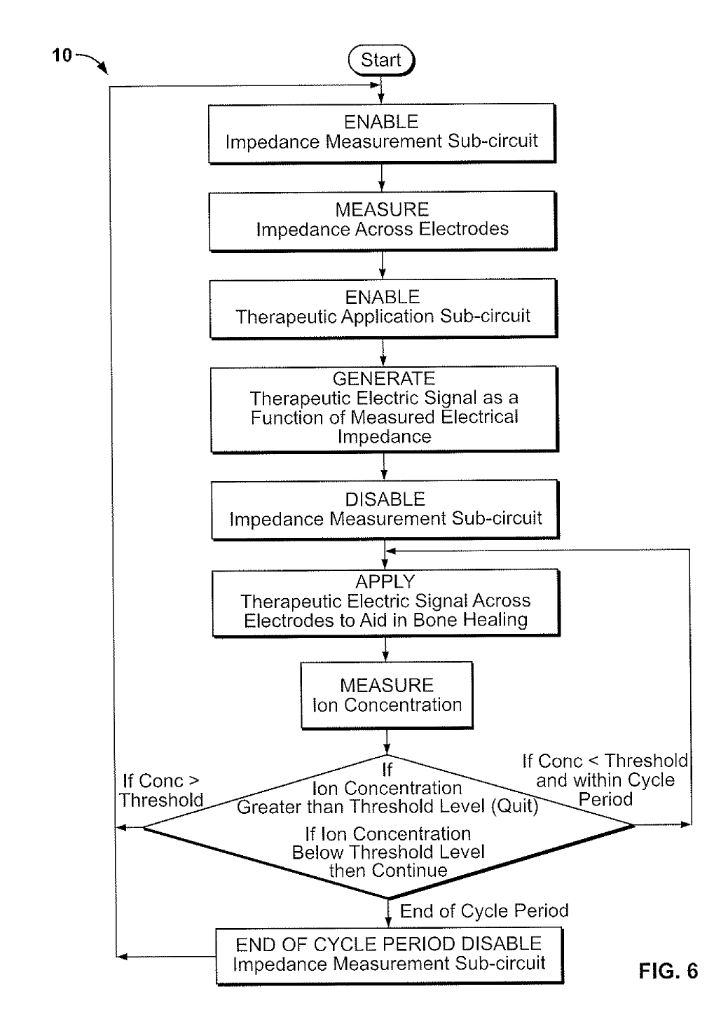

Referring now to FIG. 6 is another logical schematic embodiment of an osteogenic stimulus device of the present invention.

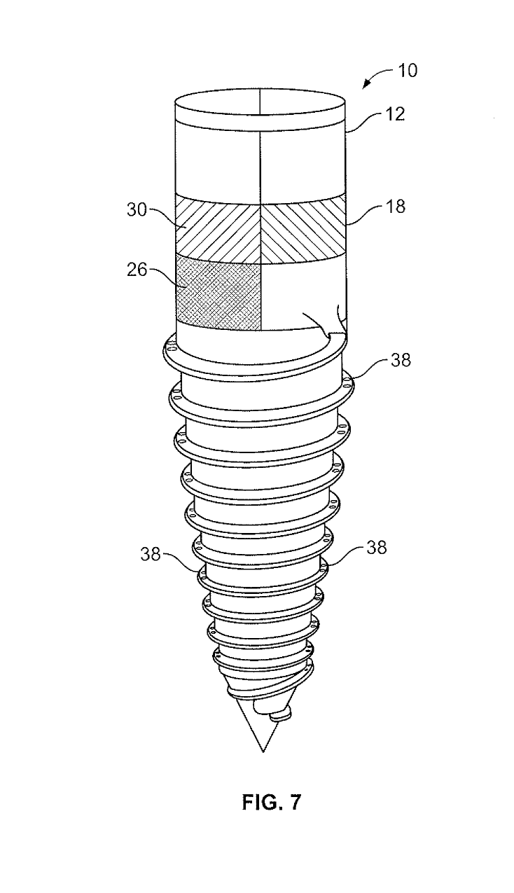

Referring now to FIG. 7 is a perspective view of another embodiment of an osteogenic stimulus device of the present invention showing a housing 12, a plurality of opposing electrode 38, a working circuit 18, an ion probe 26 and a power supply 30 in which all of these components are shown attached to the housing 12.

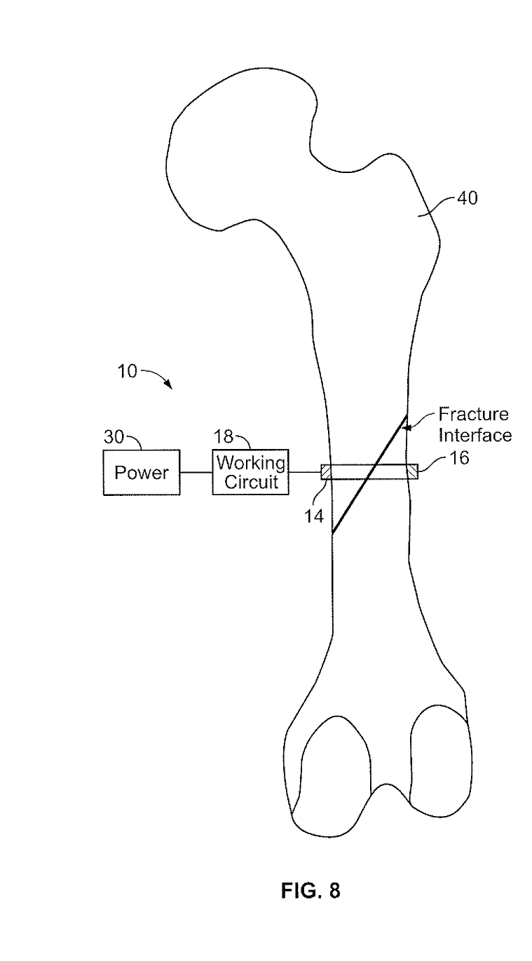

Referring now to FIG. 8 is side view of one embodiment of an embodiment of an osteogenic stimulus device mounted within a fractured bone 40 illustrating how the first and second electrodes (14 and 16, respectively) can be used to apply a therapeutic electrical signal across a fractured bone 40. Also shown are the power supply 30 and the working circuit 18 coupled to each other and to the first and second electrodes (14 and 16, respectively).

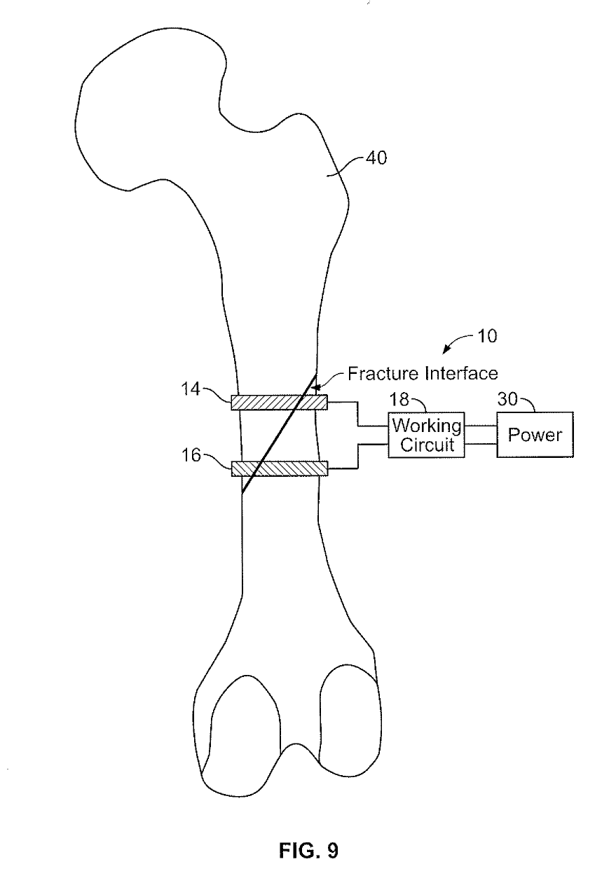

Referring now to FIG. 9 is side view of one embodiment of an embodiment of an osteogenic stimulus device 10 mounted within a fractured bone 40 illustrating that the first and second electrodes (14 and 16, respectively) can be used to apply a therapeutic electrical signal across a fractured bone along the length of the fractured bone. Also shown are the power supply 30 and the working circuit 18 coupled to each other and coupled to the first and second electrodes (14 and 16, respectively).

As to the manner of usage and operation of the present invention, the same should be apparent from the above description. Accordingly, no further discussion relating to the manner of usage and operation will be provided.

While a preferred embodiment of the osteogenic stimulus device 10 and associated methods for using the osteogenic stimulus device 10 have been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification that fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. An osteogenic stimulus device comprising:
a housing;
a first electrode attached to the housing;
a second electrode attached to the housing; and
a working circuit coupled to the first and second electrodes, the working circuit comprising:
an impedance measurement sub-circuit coupled to the first and second electrodes wherein the impedance measurement sub-circuit configured to measure an electrical impedance across the first and second electrodes;
a therapeutic application sub-circuit coupled to the first and second electrodes and coupled to the impedance measurement sub-circuit wherein the therapeutic application sub-circuit configured to apply a therapeutic electrical signal across the first and second electrodes in response to the measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit during a cycle period.

2. The device of claim 1 further comprising:
an ion probe attached to the housing; and
the working circuit further comprising
an ion monitoring sub-circuit coupled to the ion probe, the ion monitoring sub-circuit configured to measure an ion signal from the ion probe, wherein the ion signal is proportional to an ion accumulation phenomenon at an interface between the ion probe and its surrounding; and
the switch sub-circuit coupled to the ion monitoring sub-circuit wherein the switch sub-circuit configured to disable the therapeutic application sub-circuit from applying the therapeutic electrical signal when the ion signal from the ion probe is above a set threshold value.

3. The device of claim 2 wherein the ion probe is selected from the group consisting of a hydronium ion probe, a hydroxide ion probe, a calcium ion probe, a fluoride ion probe, a chloride ion probe, a potassium ion probe, and a phosphate ion probe.

4. The device of claim 2 wherein the ion probe is a hydronium ion probe and the set threshold value of the ion signal of the hydronium ion probe corresponds to a pH of about 8.

5. The device of claim 1 further comprising a power supply coupled to the working circuit.

6. The device of claim 1 further comprising the working circuit further comprises a reporting sub-circuit, the reporting sub-circuit coupled to the input impedance measurement sub-circuit, to the therapeutic application sub-circuit and to the switch sub-circuit wherein the reporting sub-circuit configured to report data information corresponding to the measured electrical impedance, to the applied electrical signal and to the cycle period, respectively.

7. The device of claim 2 further comprising the working circuit further comprises a reporting sub-circuit, the reporting sub-circuit coupled to the input impedance measurement sub-circuit, to the therapeutic application sub-circuit, to the switch sub-circuit, and to the ion probe sub-circuit wherein the reporting sub-circuit configured to report data information corresponding to the measured electrical impedance, to the applied electrical signal, to the cycle period and to the ion signal, respectively.

8. The device of claim 1 wherein the working circuit further comprises a receiver sub-circuit coupled to the therapeutic application sub-circuit and to the switch sub-circuit wherein the receiver sub-circuit configured to receive directive information corresponding to adjust the therapeutic electrical signal and to adjust the cycle period.

9. The device of claim 8 further comprising an external control system configured to be coupled to the receiver sub-circuit wherein the external control system configured to send directive information corresponding to adjust the therapeutic electrical signal and to adjust the cycle period.

10. The device of claim 1 further comprising
an ultrasonic transducer attached to the housing;
an ultrasonic sub-circuit coupled to the working circuit, the ultrasonic sub-circuit configured to drive the ultrasonic transducer to produce a sonogram signal from the ultrasonic transducer; and
an ultrasonic probe configured to receive the sonogram signal.

11. The device of claim 1 wherein the working circuit is a microprocessor working circuit.

12. The device of claim 1 wherein the measured electrical impedance is a DC resistive electrical impedance.

13. The device of claim 1 wherein the measured electrical impedance is an AC frequency dependant complex electrical impedance.

14. The device of claim 1 wherein the measured electrical impedance is a DC conductance in mhos.

15. The device of claim 1 wherein the therapeutic application sub-circuit sets a DC voltage of the therapeutic electrical signal in response to the measured electrical impedance.

16. The device of claim 1 wherein the therapeutic application sub-circuit sets a DOC voltage of the therapeutic electrical signal in response to the measured electrical impedance between 1 to 2 volts.

17. The device of claim 1 wherein the therapeutic application sub-circuit sets a DC current of the therapeutic electrical signal in response to the measured electrical impedance.

18. The device of claim 1 wherein the therapeutic application sub-circuit sets the therapeutic electrical signal in response to the measured electrical impedance between 5 to 20 microamps.

19. The device of claim 1 wherein the therapeutic application sub-circuit sets a AC voltage of the therapeutic electrical signal in response to the measured electrical impedance.

20. The device of claim 1 wherein the therapeutic application sub-circuit sets a AC current of the therapeutic electrical signal in response to the measured electrical impedance.

21. The device of claim 1 wherein the switch sub-circuit imposing a cycle period having a duration of at least one minute in which the switch sub-circuit enables the therapeutic application sub-circuit to apply the therapeutic electrical signal during at least 90% of the cycle period duration and the switch sub-circuit enabling the impedance measurement sub-circuit to measure the electrical impedance less than 10% of the cycle period duration.

22. The device of claim 1 wherein the switch sub-circuit imposing a cycle period having a duration of at least one minute in which the switch sub-circuit enables the therapeutic application sub-circuit to apply the therapeutic electrical signal during at least 99% of the cycle period duration and the switch sub-circuit enabling the impedance measurement sub-circuit to measure the electrical impedance less than 1% of the cycle period duration.

23. The device of claim 1 wherein the housing is a pedicle screw housing.

24. The device of claim 1 wherein the working circuit is attached to the housing.

25. The device of claim 5 wherein the power supply is attached to the housing.

26. The device of claim 5 wherein the working circuit and the power supply are attached to the housing.

27. An osteogenic stimulus device comprising:
a housing;
a first electrode attached to the housing;
a second electrode attached to the housing;
an ion probe attached to the housing;
a working circuit coupled to the first and second electrodes, the working circuit comprising:
an impedance measurement sub-circuit coupled to the first and second electrodes wherein the impedance measurement sub-circuit configured to measure an electrical impedance across the first and second electrodes;
an ion monitoring sub-circuit coupled to the ion probe, the ion monitoring sub-circuit configured to measure an ion signal from the ion probe, wherein the ion signal is proportional to an ion accumulation at an interface between the ion probe and its surrounding; and
an therapeutic application sub-circuit coupled to the first and second electrodes and coupled to the impedance measurement sub-circuit wherein the therapeutic application sub-circuit configured to apply a therapeutic electrical signal across the first and second electrodes in response to the measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit in a cycle period, wherein the switch sub-circuit is also coupled to the ion monitoring sub-circuit in which the switch sub-circuit is configured to disable the therapeutic application sub-circuit from applying the therapeutic electrical signal when the ion signal from the ion probe is above a set threshold value;
a power supply coupled to the working circuit.

28. An osteogenic stimulus device comprising:
a housing;
a plurality of opposing electrode pairs attached to the housing;
a working circuit coupled to each opposing electrode pairs, the working circuit comprising:
an impedance measurement sub-circuit coupled to each opposing electrode pair wherein the impedance measurement sub-circuit configured to measure a corresponding electrical impedance across each corresponding opposing electrode pair;
an therapeutic application sub-circuit coupled to each opposing electrode pair and coupled to the impedance measurement sub-circuit wherein the therapeutic application sub-circuit configured to apply a corresponding therapeutic electrical signal across each corresponding opposing electrode pair in response to the corresponding measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit in a cycle period.

29. The device of claim 28 further comprising:
an ion probe attached to the housing; and
the working circuit further comprising
an ion monitoring sub-circuit coupled to the ion probe, wherein the ion monitoring sub-circuit configured to measure an ion signal from the ion probe; and
the switch sub-circuit coupled to the ion monitoring sub-circuit wherein the switch sub-circuit configured to disable the therapeutic application sub-circuit from applying the therapeutic electrical signal when the ion signal from the ion probe is above a set threshold value.

30. An osteogenic stimulus device comprising:
a first electrode;
a second electrode; and
a working circuit coupled to the first and second electrodes, the working circuit comprising:
an impedance measurement sub-circuit coupled to the first and second electrodes wherein the impedance measurement sub-circuit configured to measure an electrical impedance across the first and second electrodes;
an therapeutic application sub-circuit coupled to the first and second electrodes and coupled to the impedance measurement sub-circuit wherein the therapeutic application sub-circuit configured to apply a therapeutic electrical signal across the first and second electrodes in response to the measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit in a cycle period.

31. The device of claim 30 wherein the first and second electrodes are pedicle screws.

32. A kit for an osteogenic stimulus device, said kit comprising:
a first electrode;
a second electrode;
a working circuit configured to be coupled to the first and second electrodes, the working circuit comprising:
an impedance measurement sub-circuit coupled to the first and second electrodes wherein the impedance measurement sub-circuit configured to measure an electrical impedance across the first and second electrodes;
an therapeutic application sub-circuit coupled to the first and second electrodes and coupled to the impedance measurement sub-circuit wherein the therapeutic application sub-circuit configured to apply a therapeutic electrical signal across the first and second electrodes in response to the measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit in a cycle period; and
a power supply configured to be coupled to the working circuit.

33. A method of using an osteogenic stimulus device to aid in healing a fractured bone, the method comprising the acts of:
obtaining the osteogenic stimulus device comprising:
a housing;
a first electrode attached to the housing;
a second electrode attached to the housing; and
a working circuit coupled to the first and second electrodes, the working circuit comprising:
an impedance measurement sub-circuit coupled to the first and second electrodes wherein the impedance measurement sub-circuit configured to measure an electrical impedance across the first and second electrodes;
a therapeutic application sub-circuit coupled to the first and second electrodes and coupled to the impedance measurement sub-circuit wherein the therapeutic application sub-circuit configured to apply a therapeutic electrical signal across the first and second electrodes in response to the measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit in a cycle period;
joining together the fractured bone;
mounting the housing to the joined fractured bone;
applying a test electrical signal across the first and second electrodes;
assessing whether or not an innervated muscle has been stimulated by the applied test electrical signal;
removing the mounted housing to the joined fractured bone when the innervated muscle been assessed to have been stimulated by the applied test electrical signal;
coupling together the power supply to the working circuit; and
activating switch sub-circuit to enable the working circuit to measure the electrical impedance across the first and second electrodes and to enable the therapeutic application sub-circuit to apply a therapeutic electrical signal across the first and second electrodes in response to the measured electrical impedance.

34. A method of using a kit for an osteogenic stimulus device to aid in healing a fractured bone, the method comprising the acts of:
obtaining the kit for an osteogenic stimulus device, said kit comprising:
a first electrode;
a second electrode; and
a working circuit configured to be coupled to the first and second electrodes, the working circuit comprising:
an impedance measurement sub-circuit coupled to the first and second electrodes wherein the impedance measurement sub-circuit configured to measure an electrical impedance across the first and second electrodes;
an therapeutic application sub-circuit coupled to the first and second electrodes and coupled to the impedance measurement sub-circuit wherein the therapeutic application sub-circuit configured to apply a therapeutic electrical signal across the first and second electrodes in response to the measured electrical impedance; and
a switch sub-circuit coupled to the impedance measurement sub-circuit and to the therapeutic application sub-circuit wherein the switch sub-circuit configured to enable alternately the impedance measurement sub-circuit and the therapeutic application sub-circuit in a cycle period; and
a power supply configured to be coupled to the working circuit;
joining together the fractured bone;
mounting the first electrode into a first section of the joined fractured bone;
securing the second electrode into a second section of the joined fractured bone;
coupling the first and second electrodes to the working circuit;
connecting together the working circuit with the power supply;
applying a test electrical signal across the first and second electrodes;
assessing whether or not an innervated muscle has been stimulated by the applied test electrical signal;
removing the first or second electrodes from the joined fractured bone when the innervated muscle been assessed to have been stimulated by the applied test electrical signal;
linking operatively together the power supply to the working circuit; and
activating switch sub-circuit to enable the working circuit to measure the electrical impedance across the first and second electrodes and to enable the therapeutic application sub-circuit to apply a therapeutic electrical signal across the first and second electrodes in response to the measured electrical impedance.
